(19) United States
(12) Patent Application Publication
(10) Pub. No.: US 2010/0234640 A1
(43) Pub. Date: Sep. 16, 2010

(54) Method for controlling multiple pollutants

(76) Inventors: Albert Calderon, Bowling Green, OH (US); Terry James Laubis, Portage, OH (US)

Correspondence Address: Albert Calderon, 1065 Melrose, Bowling Green, OH, 43402, US

(21) Appl. No.: 12/653,675

(22) Filed: Mar. 13, 2009

(51) Int. Cl. C07C 231/08; B01D 53/62; B01D 53/50; B01D 53/56

(52) U.S. Cl. 564/125; 423/351; 564/160

(57) ABSTRACT

This invention provides a comprehensive solution which comprises the production of a bed of ash-containing hot char via the pyrolysis of coal which generates a hydrogen rich gas that, subsequent to its cleanup, can be synthesized into chemicals and/or transportation fuels, while the hot char is used to: (i) reduce SO₂ and NO_{X} to elemental sulfur and to elemental nitrogen, respectively; (ii) react hot char with CO₂ to reduce it to CO, a valuable chemical that can be converted into fertilizer or into a fuel gas that can be used for electric or thermal power generation; (iii) trap particulate matter in the bed of char to join the ash in the char to form a combined ash which is eventually converted to inert slag; and (iv) filter the mercury through a portion of the char to prevent it from being emitted into the atmosphere.

### INTRODUCTION AND BACKGROUND

This invention relates to the addressing of the pollution created when electric or thermal power is generated by combusting a fossil fuel such as coal, oil, natural gas, biomass, and the like. The combustion of the fuel may be in a boiler to raise steam directly, or in a turbine used in a simple cycle or in a combined cycle configuration.

Specifically, when combusting coal in the boiler, several pollutants are produced such as particulate matter, sulfur dioxide (SO₂), oxides of nitrogen (NO_{X}), mercury (Hg), and carbon dioxide (CO₂) which was recently declared to be a pollutant by the U.S. Supreme Court. These pollutants are generally controlled by individual systems, as for example, the particulate matter is collected by means of a precipitator, the SO₂ by a scrubber, the NO_{X} by selective catalytic reduction, the Hg by activated carbon beds, and the CO₂ by a questionable system now under consideration which comprises its capture downstream of the boiler, compressing it to about 2,000 psi, and sequestering it in an underground geologic formation for permanent storage which would be continuously monitored. These various individual pollution-control systems add substantially to the capital and operating costs while at the same time increase inefficiency.

Further, the common practice for disposing particulate matter (coal-derived ash) from the precipitator is to store it in ash ponds, which creates serious environmental issues. Recently, a spill from a coal-ash pond at one of the Tennessee Valley Authority power plants covered 300 acres of land and was found to have contaminated water with arsenic. According to the New York Times of Jan. 8, 2009, there are 1,300 coal-ash ponds in the United States. It appears now that these ponds may have to be regulated.

Since around 50 percent of the electric power consumed in the United States (and about 70 percent of the power consumed worldwide) is generated with coal as fuel, there is urgency in finding a solution that will: (i) lower capital and operating costs, and (ii) address environmental concerns with respect to coal usage.

### OBJECTIVES

As will be shown herein, the main object of the instant invention is to provide a solution that specifically addresses the treatment of the above-mentioned “pollutants” in a comprehensive manner to result in a major improvement in the generation of thermal or electrical power while still using coal.

Another object of the present invention is to convert the “pollutants” into valuable by-products.

Still another object of the instant invention is to convert the “pollutants” to valuable by-products in an environmentally acceptable manner.

Yet another object of the present invention is to reduce capital investment and lower operating costs.

Further another object of the instant invention is to address the negative environmental issue of coal-ash ponds by means of said comprehensive solution.

Other objects of the instant invention will become apparent to those skilled in the art to which this invention pertains, particularly from the following description and appended claims.

Reference is now made to an accompanying drawing which forms a part of this specification wherein reference characters designate corresponding parts. It is to be understood that the embodiments shown herein are for the purpose of description and not for limiting the scope of the invention.

### BRIEF DESCRIPTION OF THE DRAWING

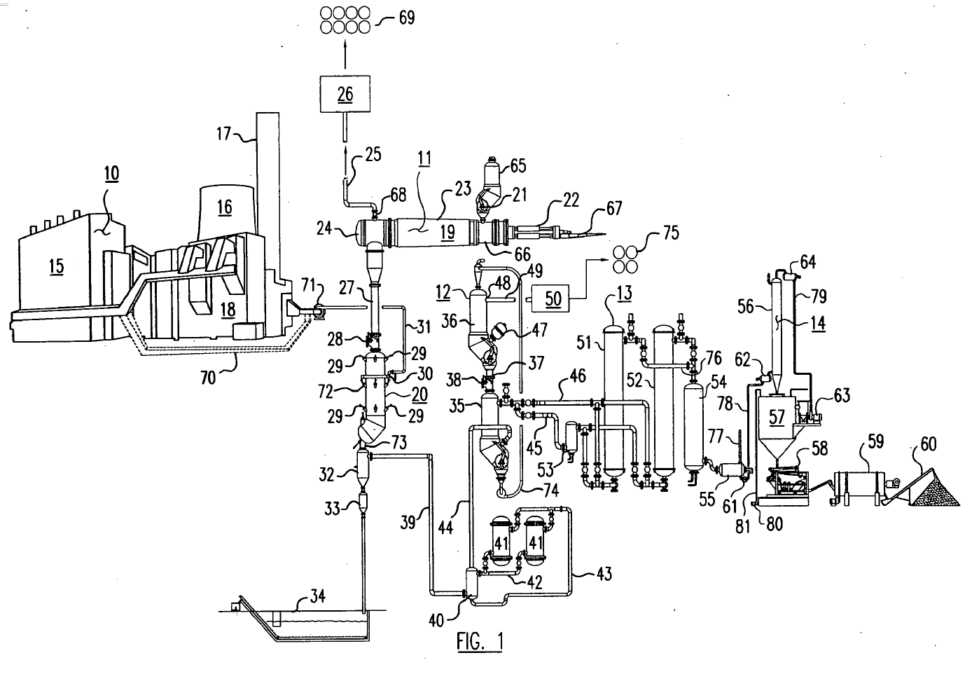

Referring to FIG. 1, numeral 10 represents a coal-burning power station, numeral 11 represents a coal converter, numeral 12 refers to a gas cleanup, numeral 13 refers to a cyanogen complex, and numeral 14 represents a fertilizer-maker.

### DETAILED DESCRIPTION OF THE DRAWING

Referring to power station 10, numeral 15 is the building within which the coal pulverizers, the boiler, the steam drum, the steam turbine and the generator are housed. Numeral 16 is the cooling tower, and numeral 17 is the stack. Power station 10 also shows a precipitator represented by numeral 18. Precipitators are commonly used in coal burning power stations.

Referring to coal converter 11, it comprises a devolatilizer denoted by numeral 19 and a reducer marked by numeral 20. Devolatilizer 19 is, in turn, equipped with coal feeder 21, charger 22, reactor 23 and discharging elbow 24 from which: (i) a hydrogen rich gas is extracted from the coal that is directed by means of duct 25 to hydrocarbon island 26 and (ii) a hot char is produced as a remnant from the coal which is fed by gravity into reducer 20 via duct 27. Reducer 20, which serves as a multi-purpose reactor, possesses a control valve denoted by numeral 28, a plurality of oxidant injection ports marked by numeral 29, and a manifold denoted by numeral 30 for the injection of flue gas containing particulate matter, SO₂, NO_{X}, CO₂, Hg and traces of other materials which result from the combustion of coal in the boiler; this flue gas originates from building 15 and is directed to reducer 20 by using duct 31. Beneath reducer 20, a slag quenching tank denoted by numeral 32 is provided to quench the slag made from the coal ash. Beneath tank 32 a lockhopper 33 is disposed in order to discharge the quenched slag into collection tank 34 which is maintained at atmospheric pressure.

Referring now to gas cleanup 12 which comprises cracker/desulfurizer 35 and sorbent regenerator 36 and both being interconnected by means of duct 37 which is equipped with control valve 38. The gas connection between reducer 20 and cracker/desulfurizer 35 is effected by means of pipe 39 connected to heat exchanger 40, thence to activated carbon beds 41 by means of pipe 42 for the removal of mercury (Hg); beds 41 may also serve to remove other pollutants such as arsenic and selenium Piping system 43 is provided to direct the gas from beds 41 to the bottom of heat exchanger 40, thence to the bottom of cracker/desulfurizer 35 via pipe 44 for cracking hydrocarbons such as tar and desulfurizing the raw gas. The gas exiting from the top of cracker/desulfurizer 35 is directed by means of pipe and valve assemblies 45 and 46 to cyanogen complex 13. Regenerator 36 is equipped with burner 47 and gas exit port 48 with pipe 49 connecting the top of regenerator 36 to sulfur plant 50.

Referring to cyanogen complex 13, it comprises reactor 51 and reactor 52 with gas-temperature moderator 53 being situated upstream of reactor 51 and gas chiller 54 being situated downstream of reactor 52. A liquefier/separator denoted by numeral 55 is disposed downstream of chiller 54, which separates the liquefied cyanogen from the unreacted gases.

Downstream of liquefier/separator 55, fertilizer-maker 14 is situated. It comprises reactor 56, settling tank 57, filter press 58, drier 59, and stacker 60. Pump 61 is provided to liquefier/separator 55 to pump the liquified cyanogen to evaporator 62, and pump 63 serves to circulate the liquid catalyst to the top of reactor 56; a heater denoted by numeral 64 serves to adjust the temperature of the circulating liquid catalyst.

### Operation

Again referring to FIG. 1 and assuming the process is running at steady state, coal hopper 65 supplies coal to feeder 21 which in turn drops a measured amount of coal into charging chamber 66, and charger 22 force feeds the coal into devolatilizer 19. An injector marked by numeral 67 injects a measured amount of an oxygen-containing gas into the charged coal, causing the combustion of a small portion of the coal under suppressed, reducing conditions, releasing a sufficient quantity of thermal energy which causes the devolatilization of the coal and thus converting the coal into a hydrogen (H₂) rich, raw gas and a hot residual char according to reaction #1.

$$Coal + {O}_{2} {{}^{suppressed}}_{combustion} C ( hot char ) + {C}_{Y} {H}_{Z} ( {H}_{2} rich gas ) \qquad ( 1 )$$

This H₂ rich gas leaves devolatilizer 19 via port 68 and is directed to a gas cleanup (not shown, but known in the art), thence by means of duct 25 it is directed to hydrocarbon island 26 where the H₂ rich gas is converted to a by-product such as a chemical like methanol which can be converted (by way of example) to gasoline or dimethyl ether with the gasoline or dimethyl ether being stored in a tank farm denoted by numeral 69. The hot residual char remaining after devolatilization is pushed out from devolatilizer 19 into the top of reducer 20 through elbow 24 with valve 28 controlling the feed to maintain a relatively fixed level in reducer 20; valve 28 also serves to maintain the pressure differential between devolatilizer 19 and reducer 20. The reactions that take place in reducer 20 comprise reactions #2(i) and #2(ii), with reaction #2(i) taking place at the top of reducer 20 and reaction #2(ii) towards the bottom of reducer 20.

4C(hot char)+2O₂→4CO at the top of Reducer 20 2 (i)

2C(hot char)+O₂→2CO at the bottom of Reducer 20 2(ii)

The flue gas resulting from the combustion of coal with air in a boiler is composed mainly of 4 parts of N₂ and 1 part of CO₂ together with some particulate matter, SO₂, NO_{X}, and Hg which are relatively small in quantity in comparison to the N2 and CO₂, but are still being considered as polluting emissions which contribute to acid rain, smog, and water contamination. When combusting coal within boiler building 15, the flue gas leaves the building in which the boiler is housed via duct 70 (shown in dotted lines) to the intake of turbo-blower 71 in order to pressurize the flue gas and deliver it to manifold 30 affixed to reducer 20, via pipe 31. The flue gas is injected into reducer 20 by circumferential injectors, one of which is marked by numeral 72. Preferably, the flue gas by-passes precipitator 18 which, in plants that already have a scrubber, may only be used as back up. In controlling emissions as discharged in this invention, the particulate matter, the NO_{X}, and the CO₂ are controlled in reducer 20, whereas the SO₂ is controlled in hot gas cleanup 12 and the Hg (with any arsenic and/or selenium) is caught in activated carbon beds 41. With respect to the particulate matter, it joins the ash in the char, and both are converted to an inert slag which runs out of 15 the bottom of reducer 20 at a temperature exceeding 2500° F.

With respect to the NO_{X} in the flue gas, it reacts with hot carbon in the char to reduce it to N₂+CO; with respect to the CO₂, which is the second largest constituent of the flue gas (the first being the N₂ when combusting the coal in the boiler with air) with a ratio of 4N₂ to 1CO₂. The following chemistry takes place within the lower half of reducer 20:

4N₂+1CO₂+C(hot char@>2000° F.)→4N₂+2CO (3)

The injection of oxygen-containing gas at the top of reducer 20 serves to cause the temperature of the hot char to rise to such an extent as to insure that all of the CO₂ contained in the flue gas injected into reducer 20 is fully reduced to CO. The injection of the oxygen-containing gas towards the bottom of reducer 20 serves to consume the carbon in the char to produce a low-Btu gas (lean gas, when using air), and at the same time melt the ash contained in the coal together with the particulate matter to forth a molten, free-flowing, vitreous liquid slag that exits through port 73 and into quencher 32 and thence through lockhopper 33 into atmospheric tank 34. Port 73, which is common for the flow of the molten slag and for the flow of the hot lean gas, insures the prevention of the slag from freezing at the bottom of reducer 20 by virtue of the elevated temperature of the lean gas being maintained above the melting point of ash. The lean gas, after merging from reducer 20, is directed to gas cleanup 12 by way of activated carbon traps 41 in order to remove vaporized Hg (and any selenium and/or arsenic), prior to the desulfurization of the lean gas in cracker/desulfurizer 35 which uses a sorbent to trap the sulfur. The sorbent, once being spent, is transported from the bottom of cracker/desulfurizer 35 by means of transporter 74 to the top of regenerator 36 to regenerate it by removing the sulfur in an elemental form vapor that is condensed in sulfur plant 50 and stored in tanks denoted by numeral 75 for export as a valuable by-product.

The lean gas, after emerging from the top of cracker/desulfurizer 35, is directed by means of piping assembly 45 to temperature moderator 53 prior to entering the bottom of reactor 51 for conversion to cyanogen (C₂N₂) which is represented by reaction #4.

4N₂+2CO(reaction #3)+6CO(reactions #2(i) & 2(ii)→4C₂N₂+4O₂ (4)

In order to prevent the O₂ from oxidizing the C₂N₂, the temperature in cyanogen reactor 51 is maintained below the ignition point of C₂N₂. The four (4) moles of C₂N₂ and the four (4) moles of O₂ are directed from the top of reactor 51 via pipe 76 to chiller 54, thence to liquefier/separator 55 in order to effect the separation of the C₂N₂ from the O₂ and from other gas that did not react. The separated C₂N₂ leaves liquefier/separator 55 as a liquid which is pumped by means of pump 61 to oxamide fertilizer-maker 14 via pipe 78. The separated O₂ is directed to the various injection points that use O₂ in the entire facility, which is represented in FIG. 1, while using pipe 77 as the original source for distribution using commonly used pumps, valves, etc., which are known in the art and therefore not shown.

The delivery of the C₂N₂ in liquid form via pipe 78 is terminated at vaporizer 62, where the C₂N₂ is converted back to a gaseous state for injection into the oxamide reactor 56, to be hydrated while the liquid catalyst is circulated through reactor 56 by means of pump 63. This liquid catalyst is preheated by means of heater 64 prior to being sprayed at the top of reactor 56, with the C₂N₂ in gaseous form, rising upwardly in reactor 56 while the catalyst in liquid form flowing downwardly in reactor 56. This intimate co-action between the two causes the efficient formation of oxamide as a thick, catalyst-containing slurry which is flushed into settling tank 57. The reaction taking place in the formation of the oxamide is according to reaction #5.

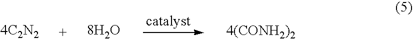

The excess catalyst in liquid form in settling tank 57 is pumped by means of circulating pump 63 to the top of reactor 56, with pipe 79 connecting pump 63 to heater 64. The thick slurry is then fed to filter press 58 where the excess liquid catalyst is pressed out of the thick slurry to be recycled, by means of pump 80, to the top of settling tank 57 using pipe 81 as a conduit. The pressed oxamide is next directed to drier 59, where it is dehydrated and thence discharged into storage, whence it is available for shipment to customers as a valuable, slow-release fertilizer by-product made from flue gas which is a waste greenhouse gas that is suspected to contribute to climate change.

It is to be noted that two C₂N₂ reactors (51 and 52) are provided in order to have the capability of having 52 as a regenerator. It is also to be noted that a system of piping and valves is also provided for the capability to remove mercury, arsenic, and selenium from the gas by activated carbon beds 41, which are adapted to switch from one to the other.

Reference is now made to the production of a by-product from the H₂ rich gas exhausted from devolatilizer 19 which, after cleanup (not shown, but known in the art), is directed to chemical by-product plant 26, where this H₂ rich gas may be utilized to make chemical by-products, one of which may be methanol that can be converted to premium gasoline to replace petroleum-derived gasoline, or another may be methanol that can be converted to dimethyl ether, a most suitable replacement for petroleum-derived diesel. Further, the H₂ rich gas is used as gaseous fuel per se or synthesized to synthetic natural gas.

All in all, it is submitted that the comprehensive solution herein disclosed provides a method for controlling multi-pollutants by processing the flue gas that results from the combustion of a fossil fuel, especially coal, used in the generation of electric or thermal power energy. This flue gas, which is considered a major polluter by containing particulate matter, SO₂, NO_{X}, CO₂, Hg and other pollutants like arsenic, is converted into valuable by-products while at the same time providing the capability to clean up a multitude of coal ash ponds which are suspected to be serious polluting sources; such cleanup comprises the reclaiming of the material in the ponds, drying it, mixing it with the flue gas and feeding the mix into reducer 20, wherein the ash is converted to a non-leaching slag.

We claim:

1. A method for treating greenhouse gases contained in a polluting flue gas which is emitted as a result of combusting a fuel and utilizing the treated gases to produce useful products comprising the following steps:
pyrolyzing coal to produce a volatile matter and a bed of hot char;
combusting a fuel to release energy while producing a polluting flue gas containing greenhouse gases;
passing said polluting flue gas through said bed of hot char to reduce at least one or more than one, oxide contained in said polluting flue gas to result in the treatment of said oxide to become a non-polluting gas;
converting said non-polluting gas into a useful by-product; and
converting said volatile matter produced in the first above-mentioned step into one or more than one, valuable by-product.

2. The method as set forth in claim 1 wherein said polluting flue gas contains a plurality of greenhouse gases.

3. The method as set forth in claim 2 wherein said polluting flue gas contains sulfur dioxide.

4. The method as set forth in claim 2 wherein said polluting flue gas contains oxides of nitrogen.

5. The method as set forth in claim 2 wherein said polluting flue gas contains carbon dioxide.

6. The method as set forth in claim 2 wherein said polluting flue gas contains particulate matter.

7. The method as set forth in claim 2 wherein said polluting flue gas contains mercury.

8. The method as set forth in claim 2 wherein said plurality of greenhouse gases comprise sulfur dioxide, oxides of nitrogen, carbon dioxide, particulate matter, and mercury.

9. A method for treating greenhouse gases existing in a polluting flue gas containing sulfur dioxide, oxides of nitrogen, carbon dioxide, particulate matter, and mercury wherein a hot char which is a reductant is used to convert the sulfur dioxide to elemental sulfur, the oxides of nitrogen to elemental nitrogen, and the carbon dioxide to carbon monoxide.

10. The method as set forth in claim 9 wherein said particulate matter is converted into inert slag by gasifying said bed of hot char.

11. The method as set forth in claim 9 wherein said mercury is recovered by means of said char acting as a carbon collecting means.

12. The method as set forth in claim 1 wherein said step of pyrolyzing coal to produce a volatile matter is further characterized by the step of cracking and desulfurizing said volatile matter to produce a syngas which is suitable to make a chemical.

13. The method as set forth in claim 12 wherein said chemical is converted to methanol.

14. The method as set forth in claim 13 wherein said methanol is converted into a transport fuel such as gasoline.

15. The method as set forth in claim 9 wherein said carbon monoxide is utilized as a fuel to generate electric power.

16. The method as set forth in claim 9 wherein said carbon monoxide is utilized as a chemical to make fertilizer.

17. The method as set forth in claim 16 wherein said fertilizer is characterized as oxamide.

18. The method as set forth in claim 17 wherein said oxamide is made from an intermediate which is characterized as cyanogen.

19. The method as set forth in claim 18 wherein said cyanogen is made from carbon monoxide.

20. The method as set forth in claim 10 wherein said particulate matter is converted into inert slag is further characterized by the step of adding other particulate matter in the form of ash and transform the combined particulate matter into an inert slag.
